(19) United States
(12) Patent Application Publication
(10) Pub. No.: US 2015/0126917 A1
(43) Pub. Date: May 7, 2015

(54) TENSIONING DEVICE FOR ORTHOSES

(71) Applicant: BAUERFEIND AG, Zeulenroda-Triebes (DE)

(72) Inventor: Gerald Stier, Langenwetzendorf (DE)

(73) Assignee: BAUERFEIND AG, Zeulenroda-Triebes (DE)

(21) Appl. No.: 14/404,864

(22) PCT Filed: Jun. 7, 2013
(86) PCT No.: PCT/EP2013/061818

§ 371 Date: Dec. 1, 2014

(30) Foreign Application Priority Data
Jun. 8, 2012 (DE) 10 2012 011 742.6

(51) Int. Cl. A61F 5/02; A61F 5/01

(52) U.S. Cl. CPC A61F 5/028; A61F 5/01
USPC 602/5

(57) ABSTRACT

A tensioning device for orthoses for supporting and maintaining the function of the human body, particularly orthoses surrounding a part of the body or the body, such as back orthoses. The tensioning device allows for an individual, segmental adjustment of the supporting effect through individually, separately designed pulley assemblies comprising at least one deflection roller and a lockable reel.

The invention relates to a novel tensioning device for orthoses for supporting and maintaining the function of the human body, particularly orthoses surrounding a part of the body or the body, such as back orthoses. The tensioning device allows for an individual, segmental adjustment of the supporting effect through individually, separately designed pulley assemblies comprising at least one deflection roller and a lockable reel.

Orthoses are therapeutic appliances for stabilizing or supporting the movement function of body parts, for example, pelvis and spinal column. Orthoses can be used directly post-traumatically, postoperatively or conservatively. As a rule, orthoses are applied around the body part, for example around the hip, and closed collar-like or belt-like such that a stabilizing pressure is applied to the body region to be stabilized. In the case of lumbar orthoses, for example, it might be necessary to immobilize a particular spinal curvature (lordosis) in order to prevent further damage to the spinal column, or to stabilize a postoperative condition so as to improve the healing process.

Known orthoses, for example lumbar orthosis belts, frequently comprise tensioning devices that can be used to increase the tension/pressure of the applied orthosis on the body part in a controlled manner. A pulley assembly device is frequently provided for this purpose. This is known to extend substantially over the entire width of the orthosis with the objective of equalizing the tensile force over the entire width of the orthosis belt. Known tensioning devices that are based on a pulley assembly have a loose pulling cable for this purpose, which runs along one side of the orthosis. It has been found that the use of orthoses designed in such a manner is fraught with disadvantages: Due to the equalizing of the tractive force caused by the known pulley assembly mechanism, an individual adjustment of the tension, and thus of the supporting effect, to an individual treatment goal is not possible. At the same time, the tensioning of known pulley assembly devices by means of one-sided pulling cables results in one-sided force action on the orthosis and thus in warping or sliding of the same.

An improved pulley assembly system is known from DE 10 2010 035 309 A1. There, multiple pulley assemblies are used. A special design of deflection rollers and/or anchoring points of the pulling cable is not provided for there.

The invention addresses the problem of further developing and improving tensioning devices for orthoses in order to avoid the known disadvantages. The technical problem addressed by the invention is that of providing an improved tensioning device for an orthosis, which can be better adjusted to the requirements of the therapy or prophylaxis, in particular with regard to the support function thereof. At the same time, the device is to be easier and more reliable to operate and adjust; sliding or twisting is to be prevented. In particular, the design of the pulley assembly system of a tensioning device is to be improved and configured in such a way that it is stable and functionally reliable, while being easy and simply to operate.

The technical problem is solved entirely by a tensioning device for an orthosis according to claim 1.

The technical problem is solved in particular by a tensioning device for an orthosis, comprising at least one pulley system, wherein the pulley system comprises at least one pulling cable and at least two pulling cable deflections, wherein at least one of the at least two pulling cable deflections is configured as a deflection roller, wherein the at least one deflection roller is mounted coaxially with a lockable reel, and wherein the reel is used to wind up and tension the at least one cable.

The tensioning device can advantageously have been or be provided in a wide variety of collars serving as orthoses, if the collars are additionally to be deliberately subjected to tension, in particular segmentally.

In a preferred embodiment, the deflection roller and the reel form part of a tensioning and deflecting element.

In a preferred embodiment, the reel has detent elements for locking the reel.

The design of a pulley system according to the invention, in which at least one deflection roller is mounted coaxially with a lockable reel for winding up and tensioning a pulling cable, and in which these are preferably designed as a tensioning and deflecting element, allows a simple, compact and easy to operate design of the pulley system and of the pulling cable deflection. The pulley system can thus be tensioned in a simple manner by rolling up or winding up the pulling cable onto the reel. As a result of the detent elements that are present, it can be ensured that the reel cannot unroll without the detent elements being actively released, whereby the pulling cable remains tensioned. The tensioning and deflecting element advantageously combines three functions, which are the deflection of the pulling cable, the tensioning of the pulling cable and thus of the pulley system, and the winding up of excess pulling cable.

In a preferred embodiment, the tensioning and deflecting element additionally comprises at least one detent element. In a preferred embodiment, the detent element comprises at least one spring mounted tooth, for example two, three or more spring mounted teeth. The detent element particularly preferably comprises two mutually opposing spring mounted teeth.

The reel preferably includes a toothed ring in which the spring mounted teeth can engage. When the spring mounted teeth engage in the toothed ring of the reel, the rotatability of the reel is preferably prevented in one direction. It may also be provided that the rotatably of the reel is prevented in both directions.

In a preferred embodiment, the tensioning and deflecting element additionally comprises at least one housing part. For example, two housing parts can be provided. The first housing part can be designed as a receiving element for the at least one deflection roller and the reel, and preferably for further elements, such as detent elements, and the second housing part can be designed as a cover for the first housing part.

In a preferred embodiment, the tensioning and deflecting element additionally comprises at least one detent element, preferably at least one spring mounted tooth, and at least one housing part.

In a particularly preferred embodiment, the tensioning and deflecting element comprises at least one deflection roller, a lockable reel, two spring mounted teeth, and two housing parts, wherein the first housing part is designed as a receiving element for the at least one deflection roller, the reel and the two spring mounted teeth, and the second housing part is designed as a cover for the first housing part.

In a preferred embodiment, the first housing part and the second housing part designed as a cover are configured in such a way that the cover can be rotated in the first housing part by a certain angle, for example an angle of 5° to 90°, in particular of approximately 10°, and that the rotation of the cover causes the same to become engaged in the spring mounted teeth and push these outwardly, so that the spring mounted teeth do not prevent the reel from rotating. If the cover is rotated by the certain angle in the other direction in this embodiment, the spring mounted teeth are not pushed apart, but engage in the teeth of the reel, so that a rotation of the reel in at least one direction is prevented.

In a preferred embodiment, the tensioning and deflecting element is made of plastic material. Preferably all the components of the tensioning and deflecting element are made of plastic material.

In a preferred embodiment, the reel has a point of application for a tool for rotating the reel and winding up and tensioning the pulling cable. The point of application can be a cross recess for applying a Philips screwdriver, for example. Other points of application that are known to a person skilled in the art are also possible, of course. The point of application can be designed in such a way that it can only be turned with a special tool. It can thus be ensured that only a person skilled in the art who is in charge, such as an orthopedic specialist who has the corresponding tool, tensions and relaxes the pulley assembly, and not by a layman, such as the patient himself or herself. It is thus possible to set an appropriate tension. As an alternative, it can also be provided, of course, that the tensioning can also be carried out directly by the patient, for example by designing the point of application as a turning knob or a turning disk.

In a preferred embodiment, all pulley assemblies of the tensioning device comprise at least one deflection roller, wherein the deflection roller is mounted coaxially with a lockable reel, wherein the reel is used to wind up and tension the at least one pulling cable.

In a preferred embodiment according to the invention, pulley assemblies are designed with multiple deflections.

A preferred embodiment is a tensioning device, containing a plurality of separate, independently tensionable tensioning segments, each having a first side element on a first end and a second side element on an opposite second end, and a central element arranged in between, wherein the first and the second side element of a tensioning segment are connected to one another by means of the central element of such tensioning segment by a first pulley assembly, or pulley assembly pair, extending between the central element and the first side element, and a second pulley assembly, or pulley assembly pair, extending between the central element and the second side element.

Special embodiments of such a tensioning device are described in DE 10 2010 035 309 A1, the content of which is hereby incorporated in the present invention. A person skilled in the art will readily combine the embodiments disclosed there with at least one pulley system according to the invention, in which at least one deflection roller is coaxially mounted with a lockable reel, wherein the reel is used to wind up and tension the at least one pulling cable, or will readily use at least one such pulley assembly in an embodiment disclosed there.

Preferred according to the invention is a tensioning device according to the invention for an orthosis, having at least one first side element on a first end and at least one second side element on an opposite second end, and at least one central element arranged in between, wherein the first and second side elements are connected to one another via the central element by means of at least one pulley assembly extending between the central element and the first side element and at least one second pulley assembly extending between the central element and the second side element, wherein the leverage points of the pulley assemblies on the central element are height-adjustable by means of detachable mechanical coupling means.

In a preferred embodiment, the first side element is connected to the central element by at least two pulley assemblies, and the second side element is connected to the central element by at least two further pulley assemblies, wherein the leverage points of the at least four pulley assemblies on the central element are height-adjustable independently from one another by means of detachable mechanical coupling means.

It has been found that the force vectors of an orthosis can be aligned individually and purposefully with the location of the pain if pulley assemblies are used which can be individually attached height-adjustably on a backplate serving as the central element, so that the pulley assemblies can deploy the greatest tensioning effect on the spinal column at the location where they are being attached on the backplate.

It has been found in particular that the force vectors of an orthosis, particularly a back orthosis, can be aligned individually and purposefully with the location of the back pain if pulley assemblies are used which can be individually attached height-adjustably on a backplate serving as the central element, so that the pulley assemblies can deploy the greatest tensioning effect on the spinal column at the location where they are being attached on the backplate. With a tensioning device according to the invention, it is therefore in particular possible to purposefully and precisely load or relieve specific spinal column sections during treatment of a patient with a back orthosis and thus achieve maximum therapeutic effectiveness.

According to the invention, the pulley assemblies are preferably attached to the central element by coupling means. According to the invention, a coupling means preferably comprises a first coupling element and a second coupling element, wherein the first coupling element is located on a pulley assembly and the second element is located on the central element.

In a preferred embodiment, the first coupling element is located on the tensioning and deflecting element. In a preferred embodiment, the first coupling element forms part of a tensioning and deflecting element. For example, the first coupling element can preferably form part of a housing part of the tensioning and deflecting element, for example by the housing part and the coupling element being integrally formed of plastic material.

According to the invention, the first or second coupling element is preferably a suspension element. According to the invention, the second or first coupling element is preferably a recess.

According to the invention, the pulley assemblies are preferably attached to the central element by mounting suspension elements of the pulley assemblies in recesses of the central element, or by mounting suspension elements of the central element in recesses of the pulley assemblies.

For example, the suspension elements can be latching elements or detent elements. The recesses, for example, can be holes. The holes can have any suitable shape, for example, round or oval. Suitable shapes are known to a person skilled in the art.

In a preferred embodiment, the detachable mechanical coupling means are designed as latching elements or detent elements.

In a preferred embodiment, the coupling elements of the central element are arranged left and right of the region covering the spinal column.

In a preferred embodiment, the leverage points of the at least one first pulley assembly and the at least one second pulley assembly are height-adjustable independently from one another. In a preferred embodiment, the leverage points of all pulley assemblies of the tensioning device are height-adjustable independently from one another.

In a preferred embodiment, the tensioning effect of the tensioning segment can be achieved by decreasing the distance of the side elements to one another by means of the pulley assemblies.

In a preferred embodiment, the pulling cables in each case run in a grid frame arranged between the central element and the side elements.

In a particular embodiment of the invention, the pulling cables of a pulley assembly are guided in a pulling cable tunnel. The pulling cable tunnel preferably extends between the side element and the central element. The pulling cable tunnel is preferably made of an elastic flexible material; an elastic knitted fabric is particularly preferred. The invention thus allows for a compact and user-friendly realization of the segmental individual pulley assemblies. This effectively prevents that contact is made with pulling cables of adjacent pulley assemblies, or entangling with pieces of clothing or other orthosis components.

In a preferred embodiment, the pulling cables of the pulley assembly run in a pulling cable tunnel which runs between the side element and central element and is made of an elastic material.

In a preferred embodiment, the tensioning device is specifically designed to be added to a knitted orthosis by way of releasable mechanical coupling means in the region of the side elements and/or of the central element.

The tensioning segments preferably each have a first side element on a first end of the tensioning device, and a second side element on an opposite second end of the tensioning device. A central element is preferably centrally arranged between the first and the second side elements. The side elements can be mechanically connected to one another by the central element, each namely by a first pulley assembly extending between the central element and the first side element, and at least one second pulley assembly extending between the central element and the second side element, said side elements thus forming a tensioning segment.

In the applied state, the tensioning device in conjunction with the orthosis is applied around the body part, and the two ends of the tensioning device are connected to one another in a force-locked manner; the tensioning device is closed similar to a belt around the body part. Within a single tensioning segment, the invention provides for at least one pulley assembly on each of the two sides of a central element, said pulley assembly extending to the side elements. The mutually opposing pulley assemblies of a tensioning segment can be tensioned in a circular manner during the use of the applied orthosis by pulling on both sides of the loose ends of the two pulling cables in opposing directions. During tensioning, the distance between the side elements, and thus between the two ends of the tensioning device, is shortened, and circular tension around the periphery of the body part is generated. Advantageously, this results in an action of force symmetrical to the central element. The circular tension can thus act directly, symmetrically and evenly on both sides of the central element, whereby the element is not displaced from the position thereof during tensioning. Twisting or shifting of the tensioning device, or of the orthosis connected to the tensioning device, is effectively prevented during tensioning.

In a departure from the prior art, the invention also preferably provides for a plurality of pulley assemblies to be arranged preferably substantially parallel to each other and independently in separate tensioning segments. Each pulley assembly engages exclusively with the associated central element and side elements of a tensioning segment. Therefore, in this embodiment, only one individually adjustable pair of pulley assemblies is associated with each individual tensioning segment. Each pulley assembly, in conjunction with the opposite pulley assembly which faces the other end, enables segmental, individually adjustable, symmetrical, circular tensioning action.

If several pulling cable deflections are provided at the one force leverage point of the tensioning segment, either on the central element or on the side element, the deflections, designed as stacked deflection rollers having a common axis, preferably form a roller block in the single force leverage point.

In a particular embodiment, the pulling cables are directly placed in the knitted fabric of an orthosis. For this purpose, the knitted fabric of the orthosis preferably has tunnel-shaped recesses or tabs, which allow the pulling cables to be guided separately. Additional measures, such as the grid frame that is provided in the previously described embodiment, for spacing apart the pulling cables of adjacent pulley assemblies are not required in this embodiment. In the simplified embodiment of a tensioning device according to the invention which is already integrated in the knitted orthosis, the ability to add or remove the tensioning device on an upright, particularly a conventional orthosis, particularly a knitted fabric orthosis, as described herein, is not required; instead, the tensioning device according to the invention is an integral component of a corresponding novel orthosis according to the invention that is described herein.

In a special variant, the respective loose ends of the pulling cables of the pulley assemblies of adjacent tensioning segments are guided into a common point of application and secured therein mutually spaced from each other.

The side elements and/or the central element preferably have connection means in order to be connected, possibly detachably, to the orthosis. The invention thus also allows the tensioning device to be added to and removed from an existing orthosis. The existing orthosis is in particular a conventional knitted orthosis, notably a rod orthosis, which due to the inherent elasticity thereof is applied around the body part and closed in the manner known per se by way of tabs.

The orthosis can in particular be a collar encompassing or surrounding a body part. The orthosis can be a back orthosis, a knee orthosis, a hand orthosis or an arm orthosis, for example. A back orthosis is preferred.

In case of a back orthosis, the tensioning device according to the invention can be attached to the orthosis in the region of the dorsal side and the hip side.

The tensioning device according to the invention allows for an individual adjustment of the tensioning force of an orthosis in a wide range. Since the stabilizing or immobilizing effect of the orthosis is essentially performed completely by the tensioning device itself, a subjacent elastic knitted fabric orthosis, which is designed in a known manner, no longer has to apply a complete tensioning or supporting effect. Advantageously, a knitted fabric can thus be used which can be used over a wide circumferential range—in case of a back orthosis, for example, both with a slender waist and with a large abdominal or chest girth—without having to provide knitted fabric orthoses which are individually adapted to the size of the body. The adaptation to the girth can preferably be made exclusively by adjusting the pulley assemblies, thus in particular by winding up or unwinding the pulling cable onto or from the reel, and the corresponding tensioning or relaxation.

The present invention also relates to a pulley assembly for use with a tensioning device for an orthosis, wherein the pulley assembly comprises at least one pulling cable and at least two pulling cable deflections, wherein at least one of the at least two pulling cable deflections is configured as a deflection roller, wherein the at least one deflection roller is mounted coaxially with a lockable reel, and wherein the reel is used to wind up and tension the at least one cable.

The present invention also relates to a tensioning and deflecting element for use with a tensioning device for an orthosis or in a pulley assembly in a tensioning device for an orthosis, wherein the tensioning and deflecting element comprises a deflection roller and a lockable reel, wherein the at least one deflection roller is coaxially mounted with a lockable reel, and wherein the reel is used to wind up and tension the at least one cable. Preferred embodiments of a tensioning and deflecting element according to the invention can be found in the corresponding description of a tensioning device according to the invention.

The present invention also relates to an orthosis, containing a tensioning device according to the invention.

The present invention also relates to the use of a tensioning device according to the invention for segmental and positionally accurate control of the tensioning or supporting function of an orthosis.

Further preferred embodiments of the invention will be apparent from the dependent claims and the additional independent claims.

Further embodiments of the present invention can be readily derived by a person skilled in the art from DE 10 2010 035 309 A1.

The invention will be described in more detail by the following drawings and description of the figures, without considering these as being limiting.

In the drawings:

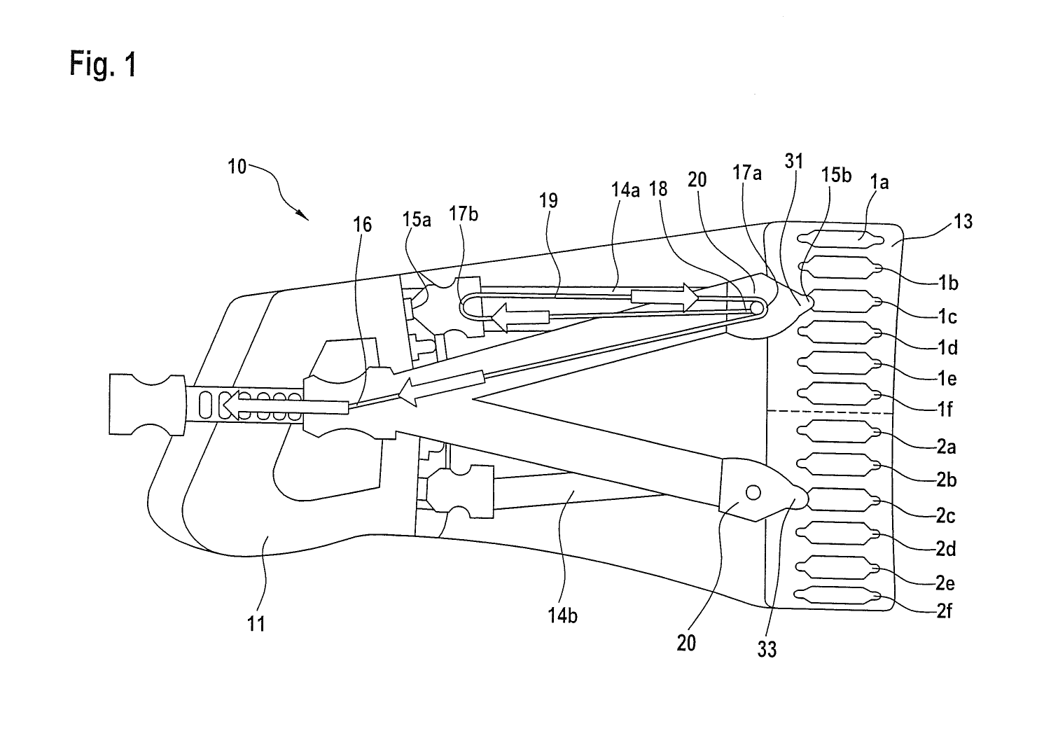

FIG. 1 shows a section of an embodiment of a tensioning device according to the invention;

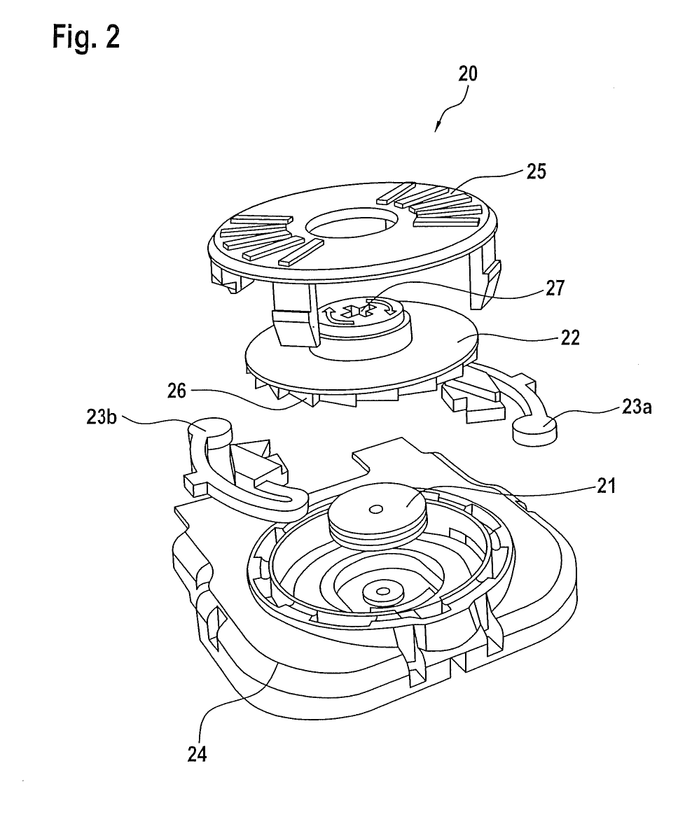

FIG. 2 shows an exploded view of an embodiment of a tensioning and deflecting element according to the invention; and

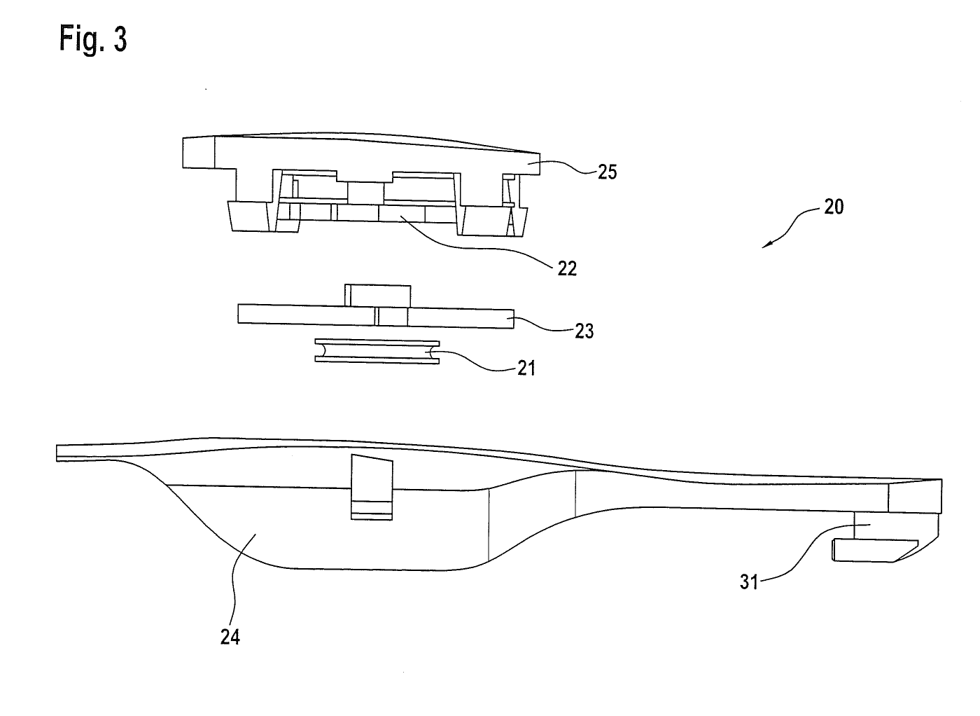

FIG. 3 shows an exploded view of a further embodiment of a tensioning and deflecting element according to the invention.

FIG. 1 schematically shows a section from a further embodiment of the tensioning device according to the invention. For reasons of clarity, the view only shows one side element (11) and the central element (13). The second side element would connect to the other half of the central element similarly to the first side element, only mirrored. The side element (11) is mechanically connected to the central element (13) by two pulling cable drives (14a, 14b) in the form of pulley assemblies. The pulley assemblies (14a, 14b) each have pulling cable deflections (17a, 17b) and pulling cable anchorings (18) for a pulling cable (19) in order to form one multiple pulley assembly each. Per tensioning segment, the pulley assemblies (14a, 14b) are thus anchored each in one force application point (15a, 15b) on the central element (13) and the side element (11). The pulley assemblies (14a, 14b) each have loose cable ends (16).

According to the invention the pulling cable deflection (17a) is designed as a deflection roller, and according to the invention the pulling cable anchoring (18) is designed as a reel, which can be used to wind up the end of the pulling cable (19) located there. The deflection roller (17a) and the reel (18) are coaxially mounted and form part of a tensioning and deflecting element (20). Such a deflecting element according to the invention is shown in more detail in FIGS. 2 and 3.

The present embodiment according to the invention is also characterized in that the leverage points, i.e. the force application points of the pulley assemblies (14a, 14b) on the central element (13) of the tensioning segments, are height-adjustable by means of detachable mechanical coupling means. The mechanical coupling means comprise elongated holes (1a to 1f, 2a to 2fb) located in the central elements (13, 23) with narrowed ends and coupling elements (31, 33) which are designed as part of the tensioning and deflecting element (20) and which can be mounted in the elongated holes (1a to 1f, 2a to 2fb) of the central element (13) by means of a latching element. The coupling element (31) can be mounted in one of the upper elongated holes (1a to 1f). The coupling element (33) can be mounted in one of the lower elongated holes (2a to 2f). Thus, according to the invention and advantageously, it is achieved that the force application points of the individual pulley assemblies (14a, 14b) are height-adjustable on the central element (13) and can therefore act in a desired region. A person skilled in the art would readily discern that a second side element can be connected to the central element (13) in the same manner.

FIG. 2 shows an exploded view of an embodiment of a tensioning and deflecting element (20) according to the invention. This element comprises a pulling cable deflection in the form of a deflection roller (21), a reel (22) for winding up and tensioning a pulling cable, two detent elements in the form of spring mounted teeth (23a, 23b), a first housing part (24), and a second housing part (25) designed as a cover. The deflection roller (21) and the reel (22) are coaxially mounted. The reel (22) furthermore has toothing (26) engaging in the toothed springs (23a, 23b). The reel (22) furthermore has a point of application (27) for a tool in the form of a cross recess.

The cover (25) can be rotated in the first housing part (24) by a certain angle, for example 10°. The cover (25) can thus engage in the toothed springs (23a, 23b) and push these outwardly. The free wheel is thereby unlocked, the reel (22) can be rotated counterclockwise.

If the cover (25) is turned back again by the certain angle, the spring-loaded teeth (23a, 23b) engage in the toothing (26). The reel (22) can be turned clockwise using a Philips screwdriver, so that a pulling cable, which is not shown, can be rolled onto the reel (22) and thus tensioned. Unrolling of the pulling cable in this position is not possible since this is prevented by the engagement of the toothed springs (23a, 23b) in the teeth (26) of the reel (22).

FIG. 3 shows a further embodiment of a tensioning and deflecting element (20) according to the invention in an exploded view. Again, the deflection roller (21), the reel (22), a detent element in the form of a spring mounted tooth (23), a first housing part (24), and a second housing part (25) implemented as a cover can be seen. The operating principle is the same as that of the tensioning and deflecting element shown in FIG. 2.

The first housing part (24) is designed in the present example in such a way that it has a coupling element (31) which can be coupled into a central element of a tensioning device according to the invention. In the present example, the coupling element (31) is designed as a latching element which can be mounted into elongated holes of a central element of the tensioning device.

1-14. (canceled)

15. A tensioning device for an orthosis comprising:
at least one pulley assembly, the at least one pulley assembly including at least one pulling cable and at least two pulling cable deflections, at least one of the at least two pulling cable deflections being designed as a deflection roller coaxially mounted with a lockable reel, the lockable reel operative to wind up and tension the at least one pulling cable.

16. The tensioning device according to claim 15, wherein the deflection roller and the lockable reel form part of a tensioning and deflecting element.

17. The tensioning device according to claim 15, wherein the lockable reel includes detent elements for locking the reel.

18. The tensioning device according to claim 16, wherein the tensioning and deflecting element additionally includes at least one detent element, and at least one housing part.

19. The tensioning device according to claim 18, wherein the at least one detent element is at least one spring mounted tooth.

20. The tensioning device according to claim 15, wherein the lockable reel has a point of application for a tool for rotating the lockable reel and for winding up and tensioning the pulling cable.

21. The tensioning device according to claim 15, further comprising a plurality of separate, independently tensionable tensioning segments, each having a first side element on a first end and a second side element on an opposite second end, and a central element arranged in between, wherein the first and second side elements of each tensioning segment are connected to one another via the central element of the tensioning segment by a first pulley assembly, or pulley assembly pair, extending between the central element and the first side element, and a second pulley assembly, or pulley assembly pair, extending between the central element and the second side element.

22. The tensioning device according to claim 20, wherein the tensioning effect of the tensioning segment can be achieved by decreasing the distance of the side elements to one other by means of the pulley assemblies.

23. The tensioning device according to claim 15, wherein the pulling cables run in a grid frame that is arranged between the central element and the side elements.

24. The tensioning device according to claim 20, wherein the pulling cables of the pulley assembly run in a pulling cable tunnel made of an elastic material, which runs between the side element and central element.

25. The tensioning device according to claim 15, in combination with a knitted orthosis, the tensioning device connected to the knitted orthosis by a releasable mechanical coupling means in a region of the side elements and/or of the central element.

26. The tensioning device according to claim 15, wherein each pulley assembly of the tensioning device includes comprise at least one deflection roller, wherein the deflection roller is mounted coaxially with a lockable reel, wherein the lockable reel is used to wind up and tension the at least one pulling cable.

27. The tensioning device of claim 15, in combination with the orthosis.

28. The tensioning device according to claim 15, wherein the orthosis is a back orthosis, a knee orthosis, a hand orthosis or an arm orthosis.

29. A method comprising:
providing a tensioning device having at leastone pulley assembly, the at least one pulley assembly including at least one pulling cable and at least two pulling cable deflections, at least one of the at least two pulling cable deflections being designed as a deflection roller coaxially mounted with a lockable reel, the lockable reel operative to wind up and tension the at least one pulling cable;
connecting the tensioning device to an orthosis; and
segmentally controlling a tensioning function or a supporting function of the orthosis.
